(19) United States
(12) Patent Application Publication
(10) Pub. No.: US 2004/0015176 A1
(43) Pub. Date: Jan. 22, 2004

(54) Stereotactic localizer system with dental impression

(76) Inventor: Eric R. Cosman, Belmont, MA (US)

Correspondence Address: Chief Patent Counsel, U.S. Surgical, a Division of Tyco Healthcare Group LP, 150 Glover Avenue, Norwalk, CT, 06856, US

(21) Appl. No.: 10/371,098

(22) Filed: Feb. 21, 2003

Related U.S. Application Data
(63) Continuation-in-part of application No. 08/772,302, filed on Dec. 23, 1996, now abandoned.
Application No. 08/772,302 is a continuation of application No. 08/303,859, filed on Sep. 9, 1994, now abandoned.
Application No. 08/303,859 is a continuation-in-part of application No. 08/263,650, filed on Jun. 20, 1994, now abandoned.
(63) Continuation-in-part of application No. 08/685,765, filed on Jul. 24, 1996, now abandoned.

(51) Int. Cl. A61B 17/50

(52) U.S. Cl. 606/131

(57) ABSTRACT

Dental-based stereotactic localizers for non-invasively inducing computer tomographic (CT) slice images of the anatomy of a patient are disclosed. The localizer includes a dental piece configured and adapted to be received in the mouth of the patient, a support structure secured to the dental piece, the support structure defining an arcuate configuration which extends to the posterior margins of the teeth of the patient thus allowing an occipital region of the head of the patient to directly contact a surface of a treatment table, and a plurality of graphic reference elements, wherein the plurality of graphic reference elements produce index information on an image scan when intersected by a plane of a scan slice, wherein the index information is used to develop a mapping of data from the image scan so as to reference the data from the image scan to a coordinate system of the dental tray.

### CROSS-REFERENCE TO RELATED APPLICATIONS

The present application is a Continuation-In-Part of U.S. application Ser. No. 08/772,302 filed on Dec. 23, 1996, which is a Continuation of U.S. application Ser. No. 08/303,859 filed on Sep. 9, 1994, which is a Continuation-in-Part application of U.S. application Ser. No. 08/263,640 filed on Jun. 20, 1994; and the present application is also a Continuation-In-Part of U.S. application Ser. No. 08/685,765 filed on Jul. 24, 1996, which is a Continuation of U.S. application Ser. No. 08/263,650 filed on Jun. 24, 1994. The entire contents of which are both incorporated herein by reference.

### BACKGROUND

1. Technical Field

The present disclosure relates to stereotactic localizer systems and, more particularly, to stereotactic localizer systems with dental impressions.

2. Background of Related Art

The field of frame-based stereotaxy and frameless stereotaxy is now actively worked on in neurosurgery. Frame-based stereotactic application is illustrated by the work of Russell Brown, Theodore Roberts, and others, and exemplified by the BRW and means of post and head screws which anchor the head ring to the skull of the patient. The head ring acts as a mechanical coordinate reference system relative to the stereotactic instrument. This frame-based coordinate system is referenced to the coordinate system of computer tomographic image slices (CT slices) by means of a localizer structure which can be attached to the head ring during CT scanning. Brown's invention of having index rods and diagonal elements included in this structure enables fiducial markings to appear on each individual CT slice, which also include the anatomy of the patient and the target region. In this way, a mapping from the individual CT slices to the stereotactic frame can be done in a precise manner, whether the CT slices are parallel to or non-parallel to the plane of the head ring.

Drs. Steven Gill, David Thomas, and Eric Cosman have developed a relocatable head frame (i.e., the GTC Relocatable Head Frame) which is analogous to the head ring described above for the BRW and CRW Stereotactic Frames, but which does not have invasive head screws anchoring the head frame to the skull of the patient. Instead, the GTC Relocatable Head Frame uses a dental impression of the teeth of the patient together with an impression of the occipital region and biasing means to secure the head of the patient to the dental impression and the occipital impression to reference the relocatable head frame to the skull of the patient by the medium of the teeth of the patient or hard palate. The GTC Relocatable Head Frame, manufactured by Radionics, Inc., may also be coupled with the CT localizer of the type described by Brown to be used in a CT scanner as described above. Again, CT slice data is referenced to the GTC Relocatable Head Frame by means of a mapping or transformation of the 2-D or 3-D data from the scanner to the coordinate system of the relocatable frame.

In the field of frameless stereotaxy, a digitized pointer or navigator such as an articulate arm, infrared light system, or magnetic tracking system is used to point at anatomical targets, for example in the head, and correlate its spatial direction to graphic images from tomographic scanning. This correlation is done, for example, by picking off identifiable points on the anatomy of the patient which are seen in the CT scanning and identifying those same physical points with the stereotactic digitizer. By touching each of these physical points, for example using the digitizer in calibration mode, the digitizer can effectively be calibrated with respect to the graphic image from the CT scanning. This process would not necessarily require putting a head ring on the patient prior to CT scanning, and thus is known as frameless stereotaxy. Typically, at least three non-colinear points would be used for fiducial points. They would be seen in the stack of CT image slices, thereby registering the slices, with knowledge of the scanner couch index, in a three-dimensional stack, and thereafter the stereotactic 3-D digitizer could be used to touch off those three points in calibration mode. The three fiducial points could be natural anatomical landmarks such as the ears, nose, etc., or they could be implanted or stuck on indicators such as radiopaque BBs, skin staples, and other markers on the skin that would appear in CT, MRI, or other tomographic imaging. The difficulty with this frameless technique is that the anatomical landmarks, if skin-based, can move and shift with stretching and movement of the skin, causing inaccuracies in the calibration and stereotactic procedure. It would be desirable to have a skull-based reference system, preferably non-invasive, for such frameless identification and calibration using the stereotactic digitizing pointer. References to frameless technology can be found in the articles of Guthrie, Adler and Watanabe et al., and also the commercial literature of Radionics, Inc. and ISG, Inc.

Anichkov et al., in their U.S. Pat. No. 4,228,799, have indicated use of a dental tray or mouthpiece attached to a platform with three vertical marker rods (10). This is used for a plane film X-ray projection to be correlated with a phantom base in older, classical stereotaxy which uses plane X-ray imaging, not tomographic imaging. No indication of its application in conjugation with indexing of CT tomographic slices or its use in combination with frameless stereotactic systems or frame-based stereotactic arc systems of the CT tomographic variety was indicated or taught in Anichkov et al.'s patent. With Anichkov et al.'s invention, it was not possible to index or reference the CT scanner slices as they are scanned through his dental-related instrument, producing a stack of two-dimensional tomographic scan slices, since there is no teaching of means to index each or any number of such 2-D slices relative to each other or relative to their mouthpiece. For example, no diagonal elements or rods are shown in Anichkov et al. which could give varying index marks on CT slices to index or map the 2D CT slices into the frame of the CT scanner or the anatomical reference frame.

Ear, nose and throat surgery (hereinafter, “ENT surgery”) frequently involves taking CT X-ray tomographic slices which are essentially parallel to the plane of the face, that is, in a nearly coronal aspect relative to the head. Typically the patient is lying prone, that is, with his stomach downward, on the CT scanning table, and his face looking into the aperture of the scanner. In this configuration, the slices are nearly coronal. It is an objective in ENT surgery to use the tomographic slices to coordinate insertion of tools into critical areas around the nose, sinuses, eyes and skull base. Great precision is required, since these are dangerous and delicate areas, where a 1 or 2 millimeter error in positioning could be harmful or fatal. Accordingly, the need exists for an improved skull-referenced indexing system. In addition, the need exists for an improved skull-referenced indexing system which would be suitable and convenient for the ENT coronal slice application.

### SUMMARY

Dental-based stereotactic localizers for non-invasively inducing computer tomographic (CT) slice images of the anatomy of a patient are disclosed. The dental-based stereotactic localizers disclosed herein include a dental piece configured and adapted to be received in the mouth of the patient, a support structure secured to the dental piece, the support structure defining an arcuate configuration which extends to the posterior margins of the teeth of the patient thus allowing an occipital region of the head of the patient to directly contact a surface of a treatment table, and a plurality of graphic reference elements, wherein the plurality of graphic reference elements produce index information on an image scan when intersected by a plane of a scan slice, wherein the index information is used to develop a mapping of data from the image scan so as to reference the data from the image scan to a coordinate system of the dental tray.

It is contemplated that the plurality of graphic reference elements are parallel to one another. It is further contemplated that the plurality of graphic reference elements are oriented in a direction parallel to the longitudinal axis.

According to one aspect of the disclosure the dental-based stereotactic localizer includes at least one index marker on each graphic reference element. It is envisioned that the index markers are radiopaque markers located at specific locations along graphic reference elements.

According to a further aspect of the present disclosure the dental-based stereotactic localizer includes at least one diagonal graphic reference element extending between adjacent parallel graphic reference elements. It is envisioned that at least one of a distal and a proximal end of each diagonal graphic reference element is connected to adjacent parallel graphic reference elements.

In one aspect of the present disclosure a distal end of each parallel graphic reference element includes a mechanical contact point. It is envisioned that the mechanical contact point is an indentation, a divot and/or a drill hole.

The dental-based stereotactic localizer further includes a stereotactic apparatus having a pointer element at a distal end thereof, wherein the pointer element is used to touch various points along at least one of the diagonal and parallel graphic reference elements to produce a mapping between a coordinate system of the stereotactic apparatus and a coordinate system of the stereotactic localizer.

It is an object of the present disclosure to provide a skull-referenced indexing system to transfer information from the reference plane of the tomographic imaging machine to a reference plane attached to the skull of the patient via a mouthpiece or dental impression device, which in turn can be used to reference the coordinate frame of a stereotactic instrument by using the pointer of said stereotactic instrument to calibrate from well defined physical points on the dental localizer system.

It is another object of the present disclosure to provide an extremely lightweight dental localizer for indexing CT slices which does not depend on a heavy head frame or head ring, but can be supported easily by the patient himself in his mouth with minimal effort or augmentation by straps.

It is a further object of the present disclosure that the dental localizer be used without need for an impression of or support from the occipital region or other portions of the head of the patient, such as the ear canals, in order to stabilize the dental piece or localizer apparatus relative to the head of the patient.

It is another object of the present disclosure to provide a simple, lightweight indexing system to reference one set of tomographic scans to another set of tomographic scans with an easily applied dental localizer structure.

It is yet another object of the present disclosure to provide a simple dental localizer that can provide reference indicia from CT scanning to the anatomy of the patient for the purpose of correlating graphic image reconstructions of the anatomy of the patient and to provide subsequent referencing of the anatomy of the patient to an external treatment apparatus or a stereotactic instrument such as a stereotactic frame, frameless stereotactic navigator, or LINAC X-ray treatment machine or other radiation therapy simulators or treatment machines so as to calibrate the external instrument or stereotactic system to the dental localizer and thus to the anatomy of the patient repeatedly and without need for invasive head screws.

It is yet another object of the present disclosure to make the dental localizer compatible with CT, MRI, and other scanning modalities so image data from these modalities can be easily correlated in a non-invasive and patient-friendly manner, without the need for heavy frame-based apparatus.

It is a further object of the present disclosure to provide a highly precise skull-based reference system for accurate localization in CT or other tomographic imaging of critical anatomy in the skull base, nose, mouth, neck, and the spine. This would be to serve the critical needs of ear, nose, and throat (ENT) surgeons, neurosurgeons, or orthopedic surgeons for dealing with approaches to critical structures in the ENT region, skull base, or spine, both interventionally or in terms o [symbol] radiographic or critical treatment methodologies.

It is another object of the present disclosure to provide a simple, accurate, noninvasive, and routinely usable device for stereotactic image data collection associated with tomographic or other image acquisition of the head and neck. This would have important implications for routine survey or diagnostic scanning of patients to immediately install the image data into an absolute reference frame relative to the skull of the patient and to remove inaccuracies associated with CT slice variation, CT couch sag and inaccuracies, improper CT scanner indexing, image distortion, patient movement and instabilities during scanning, etc.

It is a further object of the present disclosure to provide physical index points on the tomographic localizer attached to the dental impression piece which can be used to calibrate, re-calibrate, and locate stereotactic tools, radiation machines, or other instrumentation such as a microscope or ultrasonic head relative to the anatomy of the patient at the time of surgery, and all coordinated and cooperatively related to the image slice data and mapping thereof into the frame of reference of the dental impression means.

These objects, together with other objects of the disclosure, along with the various features of novelty which characterize the disclosure, are pointed out with particularity in the claims annexed to and forming a part of this disclosure.

### BRIEF DESCRIPTION OF DRAWINGS

By way of example only, preferred embodiments of the present disclosure will be described herein with reference to the accompanying drawings, in which:

FIG. 1A illustrates a stereotactic localizer in accordance with an embodiment of the present disclosure, wherein straight index rods are used in connection with a mouthpiece for target localizations;

FIG. 1B illustrates a stereotactic localizer in accordance with an alternative embodiment of the present disclosure, wherein straight index rods and diagonal rod elements are used in connection with a mouthpiece for target localizations;

FIGS. 2A-2C illustrate the relationship between CT scan coordinates, the coordinates of the stereotactic localizer of FIG. 1B, the coordinate frame of a stereotactic instrument in the operating room, and the use of computer graphic rendering to indicate the slice indexing;

FIG. 3A is a side-elevation view of the stereotactic localizer of FIG. 1B;

FIG. 3B is a side elevational view of the stereotactic localizer of FIG. 1A;

FIG. 4 illustrates the use of discrete index points located relative to the stereotactic localizer of FIG. 1A, in connection with an interoperative or treatment apparatus so as to reference the apparatus to the anatomy of the patient;

FIG. 5 illustrates a stereotactic localizer in accordance with yet another embodiment of the present disclosure in which index points located on the stereotactic localizer are used to reference a surgical instrument relative to the anatomy of the patient;

FIGS. 6A-6D illustrate various embodiments and formations of the stereotactic localizer for tomographic scanning with multiple or single diagonal elements, discrete point localization for scanning or interoperative and treatment use, and replacement localization grids attached to the stereotactic localizer;

FIG. 7 illustrates in more detail the use of target-locating or target-aligning grids or plates which can be attached to the stereotactic localizer of the present disclosure for alignment of the patient with respect to a stereotactic apparatus or a radiation treatment machine;

FIG. 8 illustrates how a stereotactic localizer can be affixed to an image scanner or treatment table by a locating or fixating bar structure so as to stabilize the position of the patient relative to the table and other external apparatus near the table;

FIG. 9A is a perspective view of a stereotactic localizer in accordance with another embodiment of the disclosure for use in ENT applications;

FIG. 9B illustrates index data as it might appear from a quasi-axial CT slice taken transversely through the anatomy of the patient and the localizer of FIG. 9A;

FIG. 9C illustrates index data as it might appear from a quasi-axial CT slice taken longitudinally through the anatomy of the patient and the localizer of FIG. 9A;

FIG. 10A is a perspective view of a stereotactic localizer in accordance with another embodiment of the present disclosure which provides indicia for tomographic slicing in a nearly coronal aspect, as well as index points for referencing a stereotactic instrument during surgery;

FIG. 10B illustrates how the index points of FIG. 10A would appear in a typical to-dimensional X-ray CT slice; and

FIG. 10C is a side elevational view of the stereotactic localizer of FIG. 10A.

### DETAILED DESCRIPTION OF PREFERRED EMBODIMENTS

The following detailed description is merely a set of illustrative embodiments of the disclosure. Accordingly, the present disclosure is not intended to be exhaustive of all possible variations of the stereotactic localizer disclosed herein.

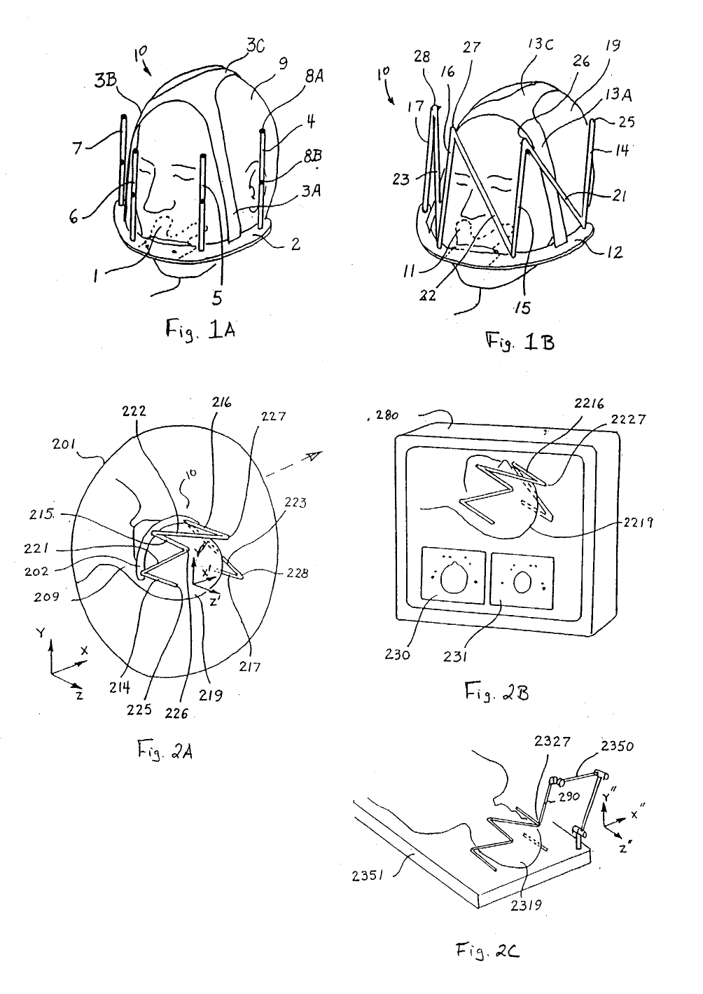

Referring to FIG. 1, a dental-based stereotactic localizer in accordance with an embodiment of the present disclosure is generally shown as 10. Stereotactic localizer 10 includes a dental tray 1 held in fixed position relative to the teeth of the patient by an impression of the teeth of the patient which is formed in dental tray 1. Dental tray 1 is attached mechanically to a support element 2 which supports index rod means 4, 5, 6 and 7. Each index rod means 4, 5, 6 and 7 may further support index markers 8A and 8B. Index markers 8A and 8B may, for example, be radiopaque markers located at specific physical locations on index rod means 4, 5, 6 and 7 and in turn located in specific physical locations relative to support element 2. Preferably, the patient bites onto dental tray 1 with his own jaw action in order to support the entire stereotactic localizer 10.

Alternatively, it is envisioned that stereotactic localizer 10 may be held in place by a strap or other securing means illustrated by straps 3A, 3B and 3C which pull up on support element 2 so that dental tray 1 is secured firmly against the upper teeth of the patient. Strap 3C can go around to the back of the head of the patient and be secured (not shown) to the rear portion of support element 2.

The patient may be imaged with stereotactic localizer 10 in place by various types of imaging devices. For example, if an X-ray-based computer tomographic (CT) scanning machine is used, the anatomy of the patient will be scanned by the CT scanner, and if the marker elements 8A and 8B are radiopaque, or indeed index rods means 4, 5, 6 and 7 are radiopaque, they will appear as index marks or fiducial markers in the CT slice, depending on the position of the slice and knowledge of the scanner. Such index information can allow the registration of the scanner image information or data from the CT scanner to be related to dental tray 1 and support element 2. Dental tray 1 and its coupling to support element 2, therefore, establish a mechanical reference frame to which the imaging data can be referred.

Turning now to FIG. 1B, stereotactic localizer 10 includes vertical and diagonal rod structures 14-16 and 21-23, respectively, which are analogous to those illustrated in U.S. Pat. No. 4,608,977 to Brown. As described in Brown, the CT scan slices which intersect these vertical and diagonal rob structures can provide an index of each individual scan slice by means of reference indicia in the scan slice itself. In this way, each scan slice can be mathematically or graphically mapped into a coordinate reference frame which is represented by dental piece 11 or by support element 12. In the case of the diagonal structures, the index marks on each slice vary as a function of the orientation and position of the slice as it intersects stereotactic localizer 10, thus providing index or reference means between the image scan data and dental piece 11.

The referencing of stereotactic localizer 10 to the dental tray is further illustrated in FIG. 2. The circular element 201 illustrates the aperture of a CT scanner apparatus. The stereotactic localizer 10 is shown in place on the head of the patient, with the head of the patient intersecting the plane defined by aperture 201 of the CT scanner apparatus. Rods 214, 215, 216 and 217 are co-linear with one another and orthogonal with respect to support element 202, which support element 202 is further attached to the dental tray (not shown). The X-ray CT slice is essentially in a plane approximately associated with the plane of aperture 201 of the CT scanner and intersects both the anatomy of the patient and vertical rod structures 214-217 and diagonal rod structures 221, 222 and 223.

The data generated by the CT scan is transferred, as illustrated by the dashed line and arrow, to a computer workstation 280 and, in this case, a computer workstation display. As seen in FIG. 2B, two of the possible CT slices are represented by images 230 and 231 on the screen. As the patient is advanced into aperture 201 of the CT scanner, the CT slices will assume different positions through the anatomy of the patient as well as through stereotactic localizer 10. The pattern of the intersection points of the CT slices with vertical rod structures 214-217 and diagonal rod structures 221-223 will show up as varying patterns on the CT slice scan image as illustrated in images 230 and 231 defining index information. The index information can be used to develop a mapping or transformation of the data of the CT slice image so as to reference the data to the 3-D coordinate system of the dental tray and its associated support element 202. One may consider the coordinate system X, Y, Z to be that of the CT scan plane (i.e., the X and Y axes) and the axis perpendicular to the CT scan plane (i.e., the Z axis) while the reference frame associated with the dental tray may be illustrated with the coordinate frame X′, Y′, Z′. A transformation may therefore be made between the data in the CT slice plane X and Y to the three-dimensional coordinate system X′, Y′, Z′, since the latter coordinate system is referenced to vertical rod structures 214-217 and diagonal rod structures 221-223 and to support element 202 by an appropriate definition. When a large volume or stack of such two-dimensional CT slices is accumulated, a three-dimensional reconstruction of this stack of CT slices may be made in the computer workstation 280 to give a rendering of the anatomy of the patient, as illustrated by 2219, and also of a simulation of the vertical and diagonal rod structures themselves, here represented as 2216 and 2217, respectively.

Stereotactic localizer 10 of FIGS. 1A, 1B and 2A-2C could be used to correct for patient movement in the CT tomographic scan or for inaccuracies or instabilities of the CT scan slices themselves. For instance, a movement of the patient between CT scan slices, such as seen in images 230 and 231, will cause a movement of the index marks seen in these images, and thus corrections to the movement can be made. Mapping of all the individual CT scan slice data to the same reference frame, for instance reference frame X′, Y′ and Z′ of dental tray 1, could similarly correct for patient movement or scanner inaccuracies or distortion effects.

Turning now to FIG. 2C, the phase of the procedure when a patient 2319 is brought to the operating room or a similar procedure theatre is illustrated. With patient 2319 on a table 2351, a stereotactic apparatus, as illustrated by the operating arm 2350, may be secured to or near table 2351 or to patient 2319. Such an operating arm 2350 may be, for example, an encoded operating arm as described by Watanabe et al. in an article entitled “An articulated neurosurgical navigation system using MRI and CT images”, IEEE Trans Biomed Eng, 35:147-152, 1988, and described by Guthrie and Adler in an article entitled “Computer-assisted pre-operative planning, interactive surgery and frameless stereotaxy”, Clinical Neurosurgery, 38:112-131, 1992. Such an arm is marketed by Radionics, Inc., Burlington, Mass. Operating arm 2350 will have its own reference coordinate system X″, Y″, Z″, which may be referenced to its base or some portion of the operating arm itself.

A pointer element 290 at the end of operating arm 2350 may be a surgical tool, a passive pointer, or some other device which can be used to probe the anatomy of patient 2319. Pointer element 290, in a calibration phase, may be used to touch various points such as point 2327, corresponding to the top of one of the diagonal and vertical intersection points of stereotactic localizer 10 shown in FIG. 1B. Point 2327, as seen in FIG. 2A for example, might correspond to a physical point 226 of stereotactic localizer 10. By touching off physical points on stereotactic localizer 10 with operating arm 2350, a mapping or reference can be made in this calibration maneuver between the coordinate system of operating arm 2350, as illustrated by X″, Y″, Z″, and the coordinate system of stereotactic localizer 10, as illustrated by X′, Y′, Z′ in FIG. 2A. In this way, once the calibration procedure has taken place, operating arm 2350 may then be calibrated relative to the anatomy of patient 2319 via the mappings or transformation illustrated in FIGS. 2A, 2B and 2C.

Thereafter, as pointer element 290 is moved into the space of the anatomy of patient 2319, the correspondence between the physical point of pointer element 290 and the anatomy of patient 2319 can be illustrated-by a point in the graphics display of computer workstation 280. Thus, moving operating arm 2350 in the environment of the anatomy of patient 2319 can produce on computer workstation 280 a representation of the position of pointer element 290 and point 2327 moving within the space of the virtual representation of the anatomy of patient 2219 (FIG. 2B) or as a point moving within the two-dimensional images, as illustrated by images 230 and 231. This process has been described using skin-attached or bone-attached fiducial points in the article by Guthrie and Adler. Previously, fiducial points such as X-ray-detectable markers or natural landmarks were used on the skin of the patient or the anatomy of the patient directly, and such a calibration procedure as just described for an operating arm 2350 was made by touching said anatomy-referenced fiducial points and thereby providing a mapping between the image data from the CT scanner and the coordinate system of the operating instrument. Typically, such procedures were inaccurate because of the movement of the skin of the patient, the movement of the patient within the imaging machine itself during the scanning process, and, in other cases, having to do with the inaccuracy of the scanning machines themselves with respect to indexing of the scan slices relative to the scanner couch. By using a dental-related reference means, such as those illustrated in the present disclosure, a very accurate reference can be made via the bony anatomy of the teeth directly to the skull of the patient. This kind of reference has been illustrated by the GTC Localizer of Radionics, Inc. and known to be highly accurate.

One of the novel features of the present disclosure is that, in addition to using a dental-referenced localizer structure, it provides both the localizer structures to calibrate the image data and also the contact points such as point 2327, which are connected to dental tray 1, so that an instrument such as operating arm 2350 may touch these physical reference points to provide the necessary calibration data. Because the localizer structure and the index points are connected to the anatomy of patient 2319 by dental tray 1, a much more stable and reliable transfer or mapping of the data between the scanner coordinate system, the dental coordinate system, and the operating or treatment instrument can be made.

Thus, the present disclosure solves an essential problem of frameless stereotaxy, namely, the problem of accurate calibration of the stereotactic external apparatus relative to the anatomy of the patient. By avoiding the intermediate step of referencing to the skin of the patient and by providing vertical and diagonal index rod structures 214-217 and 221-223, respectively, the entire stack of imaging data can be accurately related via the bony anatomy to the external stereotactic or treatment apparatus. One of the desirable features of the present disclosure, therefore, is to provide a reference structure to index the data of the imaging scanner to a dental tray and also to provide physical points on the structure with a known relationship to the dental tray, which can be mechanically touched by an external apparatus so as to calibrate the external apparatus to the physical contact point and therefore to the dental tray. By this contacting procedure, the reference of the external apparatus can be made to the dental tray and, therefore, to the imaged anatomy of the patient. This is an important implementation in the field of “frameless” stereotaxy in which non-invasive image data acquisition is made and subsequently a patient is brought to a procedure theatre, such as the operating room or linear accelerator (LINAC) table, and a calibration must be made of the anatomy of the patient by an external treatment apparatus such as a stereotactic instrument or a LINAC which delivers a radiation beam.

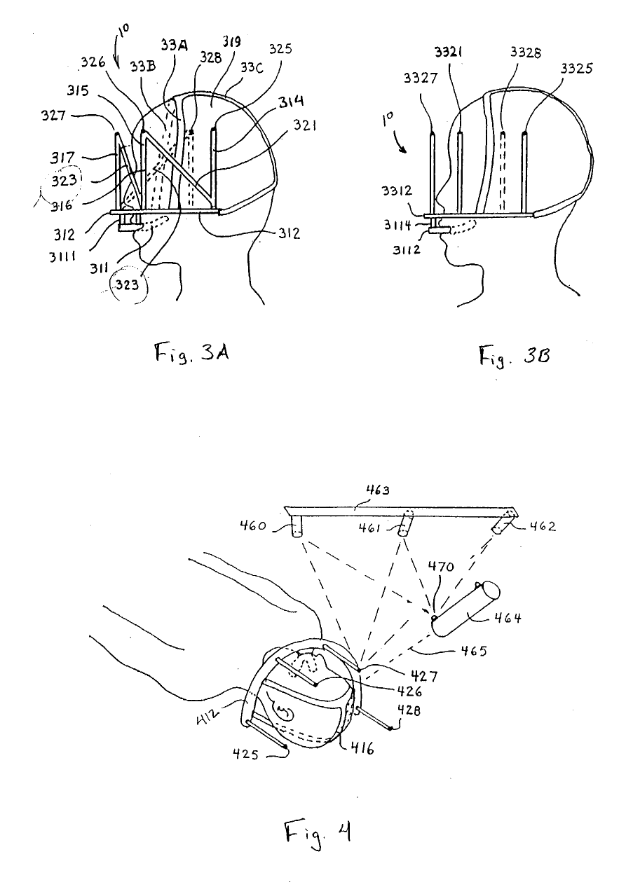

Turning now to FIG. 3A, a side elevational view illustrating further detail of stereotactic localizer 10 of FIG. 1B, is shown. As seen in FIG. 3A, dental tray 311 is secured to support element 312 by attachment member 3111. Support element 312 is further connected to vertical index rod means 315, 316, 317 and 318 with associated diagonal index rod means 321, 322 and 323. The top of each vertical index rod means 315-318 includes definable physical points 325, 326, 327 and 328, which may serve as mechanical contact points for the external stereotactic device. These mechanical contact points may be, for example, indentations, divots, drill holes, or other receptacles or mechanical attachment elements which could stabilize a probe near to or there onto so as to provide the requisite physical reference points relative to the dental tray. Mechanical contact points may also be proximity points such as metal, coil, semiconductor, or other elements which can be detected by electromagnetic (including optical), ultrasonic, or other means for a proximity tip on the probe so that actual physical contact need not be made, but rather a proximity detection contact could be made by a probe or instrument so as to localize these physical index points.

Vertical index rod means 315-318 and diagonal index rod means 321-323 may be hollow tubes, such as plastic tubes, that can be filled with MRI visible fluids, like copper sulphate solutions, so that MRI scans will detect them as index or fiducial marks in a tomographic reconstruction slice. Thus, the self-same localizer can be used to reference CT to MRI scans, or can be used to reference or cross-reference these scan modalities by themselves. If a radiation solution is put into the hollow tubes making up the index rod means, they could also be used for PET scan referencing.

Turning now to FIG. 3B, a side elevational view illustrating further detail of stereotactic localizer 10 of FIG. 1A, is shown. In FIG. 3B, a straight vertical rod adaption to dental tray 3112, which could be identical to dental tray 311 of FIG. 3A is shown. Dental tray 3112 is attached to support element 3312, in a prescribed position relative to dental tray 3112. Thus, location points 3321, 3325, 3327 and 3328, at the ends of the vertical rods, are in a known position relative to support element 3312 and thus to dental tray 3112. As a result, location of points 3321, 3325, 3327 and 3328 are in a known position relative to the geometry of stereotactic localizer 10 of FIG. 3A, or themselves could be self-standing localizers for use as identifiable radiopaque points relative to the anatomy of the patient. The stereotactic instrument thereafter could touch these location points so as to calibrate the instrument relative to the anatomy of the patient. The present disclosure encompasses the set of instruments, including dental piece 311 together with CT stereotactic localizer 10 shown in FIG. 3A for indexing the CT scan slices with diagonal index rod means 321-323, as well as CT stereotactic localizer 10 shown in FIG. 3B, which has definable location points 3321, 3325, 3327 and 3328, or proximity points, so as to calibrate a stereotactic apparatus relative to the anatomy of the patient based on the registrations determined using either of the CT stereotactic localizers above.

FIG. 4 shows a use of dental-attached stereotactic localizer 10 of FIGS. 1A and 3B in an application involving an external treatment apparatus 464, which could be, for example, a stereotactic probe holder, a linear accelerator (LINAC) for delivery of radiation, or other treatment or therapeutic device. A probe or beam direction emanating from treatment apparatus 464 may be illustrated by dashed line 465. In FIG. 4, dental-attached stereotactic localizer 10 is held in place by the teeth of the patient, and index or calibration points 425, 426, 427 and 428 are held via support element 412 in a fixed position relative to the skull of the anatomy of the patient. Index points 425-428 may be contacted or identified by a portion of external treatment apparatus 464, in this case, a set of cameras 460, 461 and 462 suspended on a bar 463. Cameras 460-462 are configured and adapted to monitor the position of index points 425-428 and can either detect points 425-428 as objects in the field or, if cameras 460-426 are, for example, infrared detecting cameras, can detect index points 425-428, if index points 425-428 are sources of infrared light such as LED light emitting diodes.

A strap 416 can be provided to help maintain the dental-attached stereotactic localizer secured to the teeth of the patient so as to be, self-supporting on the teeth or to encourage the patient to hold the dental piece in a good contact position relative to his dentition. By tracking the position of index points 425-428 with cameras 460-462, as shown in FIG. 4, the position of the anatomy of the patient can thus be registered in space, and if cameras 460-462 are cooperatively coupled in a known orientation or position relative to external treatment apparatus 464, then external treatment apparatus 464 and the associated probe or beam 465 may also be known precisely relative to the anatomy of the patient. Thus, registration of external treatment apparatus 464 relative to the anatomy of the patient via index points 425-428 is one embodiment of many possible ways of achieving the same.

As further seen in FIG. 4, cameras 460-462 are configured and adapted to monitor the position of multiple or single LEDs 470 which are disposed on external treatment apparatus 464 so as to track external treatment apparatus 464 simultaneously with the tracking of the anatomy of the patient. Also, the dashed lines emanating from cameras 460-462 illustrate the light beams that communicate between cameras 460-462 and, for example, index points 425-428 on dental-attached stereotactic localizer 10 and LED 470 of external treatment apparatus. Such tracking systems are now known in the commercial industry and medical industry for locating structures in and around the anatomy of the patient.

FIG. 5 shows use of a stereotactic localizer 10 similar to that of FIG. 4 in which a head of a patient is held in position by a head clamp 570, which head clamp 570 is secured to an operating table 571 by securing means 572. Securing means 572 can be a bar, a clamp, and/or other such device. Stereotactic localizer 10 includes index points 525, 526, 527 and 528, which are secured to support element 512 which is in turn further secured to a dental tray (not shown) in the mouth of the patient. Cameras 560, 561 and 562 are configured and adapted to locate and track index points 525-528, for example as shown by the dashed lines. As seen in FIG. 5, a surgical probe 590, for example, a navigation probe or a surgical tool or instrument, has attached thereto infrared lights or other such markers 580 and 581, which can also be tracked by cameras 560-562. In this way, the position of probe 590, relative to the anatomy of the patient, can be determined by cameras 560-562 tracking the relative position of index points 525-528 and markers 580, 581 on surgical probe 590 itself. By sequentially using surgical probe 590 to touch off index points 525-528, one can, in essence, calibrate surgical probe 590 relative to the anatomy of the patient via the dental localizer, and thereafter all positions of surgical probe 590 relative to the anatomy of the patient can be known mathematically and displayed on a computer graphics workstation so as to guide the surgeon or operator to the anatomical structures within the interior of the head or neck of the patient.

It is included as part of the present disclosure that the dental-based CT or computer tomographic localizer by itself may have a unique advantage and capability, even if it is not used in conjugation with additional index points for referencing a stereotactic apparatus. That is, there is considerable utility and importance to have a dental-based localizer that can easily index CT, MRI or other tomographic imaging in two dimensions or three dimensions. The stereotactic localizers, for example shown in FIGS. 1A and 1B, can be made very lightweight and may be supported by the patient alone using his own bite force or with a minimal amount of ancillary encouragement or support, such as with straps over his head. The stereotactic localizer would have utility in registering scan imaging to avoid, for example, movement of the patient in the scan from one slice to the next, aberrations in the slice indexing due to inaccuracies of the scanner couch or gantry, uncertainties in slice thickness and separation between slices, distortion within the imaging machine itself, etc. Thus, in accordance with the present disclosure the dental localizer with CT tomographic indexing means by itself. In one embodiment, the stereotactic localizer can be made extremely lightweight, such as, for example, from plastic or carbon fiber material, such that the patient can easily hold the stereotactic localizer within his mouth when he is lying in any orientation by a lightweight bite on the dental piece. Lightweight elastic straps over the head of the patient could also be used to hold a very lightweight dental-based localizer in the mouth of the patient. The stereotactic localizer according to the present disclosure would not rely on a heavy stereotactic base frame as is used, for example, in the BRW Localizer or which, until this time, has been used with the GTC Gill-Thomas-Cosman Relocatable Frame referenced above.

As shown in FIGS. 1A, 1B and the other figures, it is envisioned that a stereotactic localizer in accordance with one of the embodiments of the present disclosure could have a support element 2, which is not a complete ring around the head of the patient but rather a plate which is semi-circular or does not extend back, in a lateral view, further than the posterior margins of the teeth of the patient. In this way, support element 2 could be made extremely lightweight. Preferably, support element 2 is not in a ring form or a frame form, but rather a curved plate structure. Thus, the present stereotactic localizer can be made very lightweight, self-supportable by the patient, head-ring free, and occipital support free. No complete ring around the head of the patient is needed because of the light weight of the stereotactic localizer. Alternative structures such as tubular or rod structures or hollow truss type structures could also be imagined which would make the stereotactic localizer both strong and lightweight for the intended purpose. As seen in FIG. 1B, diagonal rod structures 21-23 act as trusses to make the stereotactic localizer self-contained, self-supporting, lightweight, and strong.

In conventional stereotactic systems to date, tomographic localizers are typically very rugged and are heavy constructions which envelop the head of the patient and which may weigh a half pound or more. In contrast, the present disclosure, which is directed to a dental-based stereotactic localizer, could be very lightweight, on the order of a few ounces or less, and be held by the patient alone merely by biting on the dental tray. Biting and self-support of the dental-based stereotactic localizer could easily be sustained by the patient during the time of a tomographic scan, generally about several minutes. The present disclosure includes the use of such dental-based stereotactic localizers and can include the situation in which one does not need an impression of the occipital region, as is suggested in the Gill-Thomas-Cosman (GTC) Relocatable Head Frame. In the GTC Relocatable Head Frame, a substantial base ring or frame is used, and occipital impression as well as a dental impression is used together with biasing straps to pull the occipital and dental impressions to the head of the patient so as to secure the base frame or base ring in a relocatable position. The present disclosure further includes an embodiment where no occipital impression is made, but rather only a dental impression together with a stereotactic localizer is involved. The stereotactic localizer, as shown in FIGS. 1A and 1B, can either be discrete image-visible points or lines, or other geometric structures, or may include diagonal elements which enable indexing of the slice in a way illustrated by the localizer of Brown cited above, which is used relative to a stereotactic frame and head ring.

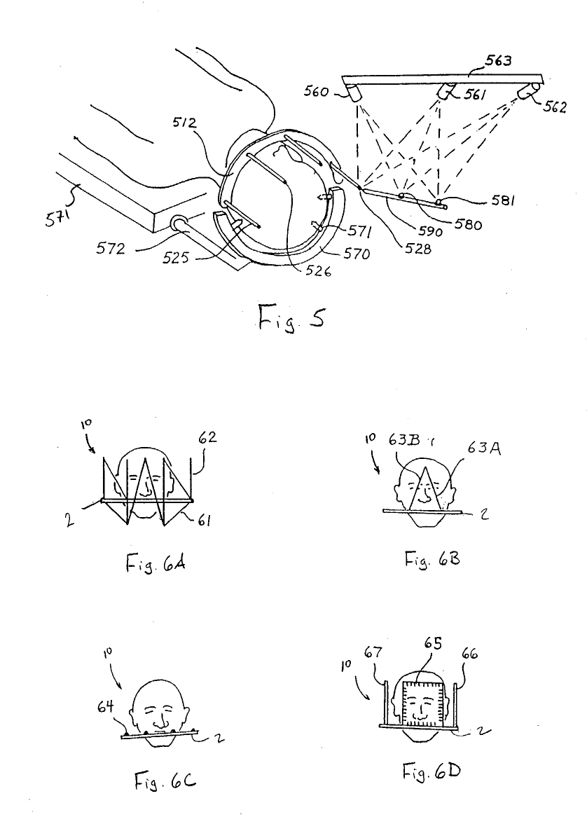

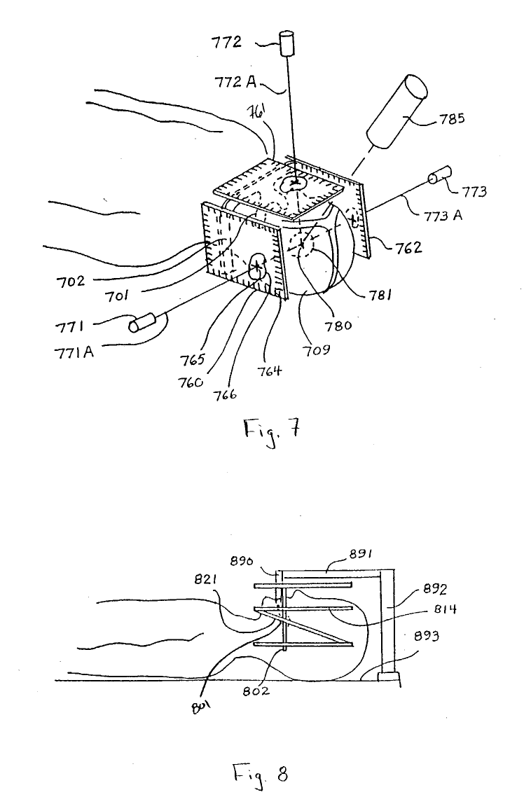

Many variations of the dental-based stereotactic localizer are also included within the scope of the present disclosure. FIGS. 6A-6D illustrate some variations, and those skilled in the art can think of many more. As seen in FIG. 6A, dental-based stereotactic localizer includes a structure of vertical and diagonal rods supported by support element 2 which is in turn supported on the dental tray (not shown). The rods extend above the approximate plane of support element 2, via, rod 62, and also extend substantially below that plane, via, rod 61. In this way, calibration indexing or mapping of the tomographic or other imaging data can be done not only for the cranium and skull base but also for the nose, jaw and cervical areas, including the spine.

As seen in FIG. 6B, the dental-based stereotactic localizer includes a reduced number of diagonal elements supported by support element 2. In the present embodiment the diagonal elements 63A and 63B form a triangle in the front of the stereotactic localizer. This single triangle may be used to index the two-dimensional CT slices, for example, given the assumption that the CT slices are parallel to some other anatomical or physical plane. This would be sufficient to reference or map the CT slices relative to the dental tray and thus the anatomy of the patient. Thus, included in the scope of this invention are graphic reference means or localizing structures which have less than three diagonal elements and may include two or only one diagonal element to provide an indexing or reference means of tomographic imaging through the localizer and the anatomy so as to reference the image data to the anatomy of the patient.

In FIG. 6C a very simple dental-based stereotactic localizer is shown. According to the present embodiment, the stereotactic localizer includes a series of discrete, observable points such as point 64, which could be visible in a CT or MRI scan, or other type of scanning such as angiogram, PET, etc provided on support element 2. A series of one or more such visible points 64 may enable a referencing of the image scan data to the dental tray (not shown) and the anatomy of the patient. Also, such a simple stereotactic localizer having fiducial contact points, such as 64, could be used during an operation or treatment to be touched or referenced by a stereotactic pointer or navigator (via arm, optical, etc.) or by a stereotactic microscope (by focusing the focal spot on the points).

As seen in FIG. 6D, the dental-based stereotactic localizer includes reticule or grid plates 65, 66 and 67 attached to support element 2 which is in turn supported by a dental tray (not shown). Grids or plates 65-67 could have various scales, index marks, which could be radiopaque such that performing a plane film or angiographic image of the anatomy would enable calculation, mapping, or referencing of the image data to the anatomy of the patient via the stereotactic localizer. This could be useful in doing angiographic or X-ray studies of the head of the patient. Such a reticule or grid system could also be used in conjunction with the diagonal or index point localizers described above for subsequent therapy such as with a linear accelerator X-ray beam. In such situations, after the tomographic scanning, using, for example, a diagonal type localizer, the grids or plates 65-67 could be put onto support element 2 in a known position and the laser lights which indicate the beam position from the linear accelerator could be shone upon the reticule or grid system so as to line up a specific anatomical point determined from the image scanning at the isocenter of the linear accelerator based on the laser light positioning.

Thus, the set of instruments included in this disclosure can include not only a support element having a dental tray and imaging localizer but also reticule systems for aligning and positioning external treatment apparatus such as radiation sources to bombard a target which has been calculated or determined by means of the imaging system with the associated localizer.

FIG. 7 further illustrates how reticule or target grid plates 760, 761 and 762 could be alternatively attached to the dental tray. In FIG. 7, a dental tray 701 is placed in the mouth of the patient, and the patient can either bite down on to dental tray 701 to secure dental tray 701 in place or straps may be used to hold dental tray 701 in place. A support element 702 may be the same as support element 2 of FIG. 1, or it can be a separate support element that attaches to the dental tray, as described above. A target may have been determined using the imaging data from stereotactic localizer 10 of FIGS. 1A and 1B, and the other embodiments shown above, whereby the coordinates of the target can be determined relative to the dental tray. The target may be some anatomical structure or pathological object such as a tumor in the head or neck of the patient. The target coordinates, for example, in the coordinate frame X′, Y′, Z′ of the dental tray and localizer, as described above, may then be set as target positions on grids 760, 761 and 762. Grids or grid plates 760-762 may be attached to dental tray 701 via support element 2. Grids or grid plates 760-762 may have millimeter or other distance markings on them, for example, as illustrated by scales 764 on plates 760. In this way, the coordinates X′, Y′, Z′ of a target calculated using stereotactic localizer 10 and from the imaging data of the anatomy may be indicated, referenced, or set upon such grid plates. Grids 760-762 need not be actual plates with scales on them, but could be, for example, translation means for a cross-hair or “bomb site” on rails or sliders which are also attached to the dental tray.

Thus, a target position, as illustrated by cross 765, and even the extended area 766, may be demarked on plate 760, as illustrated in FIG. 7. Target position 765 and area of demarcation 760 may be the projection of a centroid and an anatomical target within the body and may result, for example, from a treatment planning analysis of the imaging data set as derived from the dental tray with stereotactic localizer 10 being scanned by the tomographic scanner. Once such target settings or target demarcations have been made, as illustrated in FIG. 7 (and this may be done for one, two, three, or more plates, grids, or slider devices) then external registration means may be used from an external treatment radiation apparatus, for example, such as a linear accelerator (LINAC) so as to align the radiation beam of the external treatment irradiation device to intersect the anatomical target within the head of the patient. This is illustrated in FIG. 7 by use of the lasers 771, 772 and 773 with associated laser beams, scan laser beams, or laser sheets of light 771A, 772A and 773A, which are pre-aligned with the external treatment radiation apparatus so that they will intersect with the direction of the external radiation source or radiation beam delivery system 785. Thus, the intersection point 780 can be made to coincide with the anatomical target whose coordinates X′, Y′, Z′, for example, are set on the grid plate by translating and orienting the patient so that the laser beams 771A, 772A and 773A play on the respective target position such as 765 for one of the side plates. When this condition pertains, the beam from radiation beam delivery system 785 will intercept the anatomical target as calculated from the imaging data. The patient can easily be translated on standard couches so that the laser beams can be aligned in the way just described.

Thus, the disclosure includes the use of a dental tray with graphic localizer means as described above in conjunction with target-setting or target-localizing means which can be used in conjunction with external apparatus to align the external apparatus to a target calculated by means of the imaging data from the tomographic slices through the anatomy and the graphic reference means.

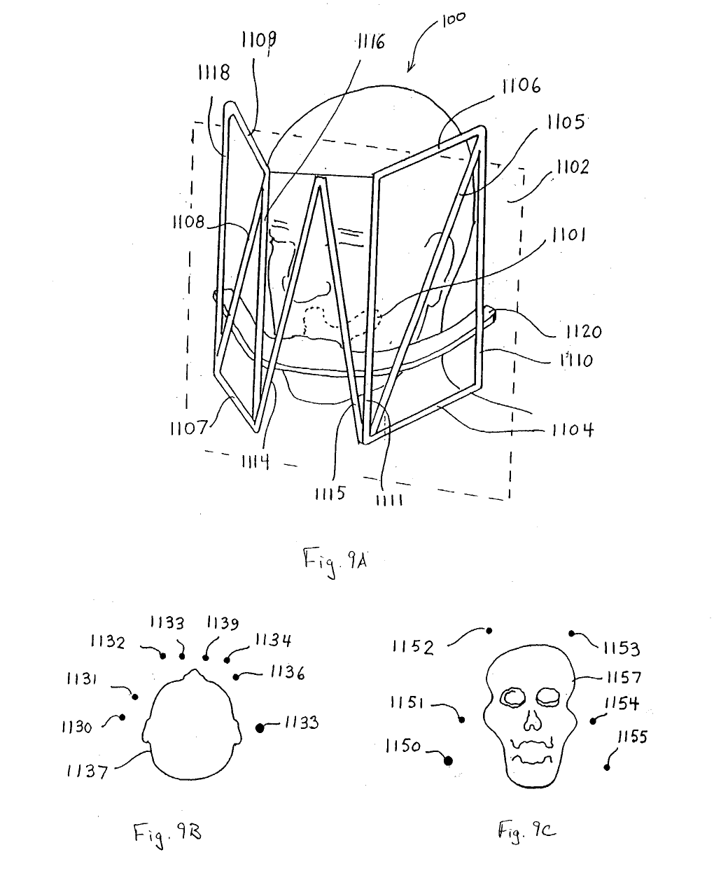

FIG. 8 illustrates how the dental-based stereotactic localizer, when retained in the mouth of the patient, can also be stabilized and supported by a support bar or other structure means so as to orient the head of the patient in a desirable or repeated orientation relative to a treatment couch or couch for an imaging scanner. As seen in FIG. 8, the patient would, for example, be lying on couch or table 893 and have dental tray 801 placed in their mouth. Vertical rod structures 814 and diagonal rod structures 821 are in place, as described above, and attached to dental tray 801 via support element 802. A connection means 890 would connect dental tray 801 to a horizontal bar 891 and further on to a support 892, which would be secured to table 893. This mechanical structure could be rigid, and therefore the orientation of dental tray 801 and therefore the anatomy of the patient relative to couch 893 could be stabilized in a specific or a fixed orientation.

In this way, the slice information from the imaging scanner would be oriented essentially in the same parallel orientation for each slice. Furthermore, if the patients were put back on the table at a later time or put onto another table, for example, in a treatment or stereotactic room or machine, the same table fixation device, including horizontal bar 891, support 892 and couch 893 could be used so as to orient the patient in the same relative position relative to the treatment or stereotactic machine. Thus, the dental tray can be used in conjugation and cooperation with other mechanical devices so as to orient the patient on a table and keep the patient in that orientation during scanning and treatment.

The importance of the present disclosure pertains still, since the localizer structure or graphic reference means in FIG. 8, being vertical rod structures 814 and diagonal rod structures 821, can be used to accurately index each slice relative to the anatomy and to be sure that each slice is in its proper position and that the patient has not moved. Furthermore, the grids, as shown in FIGS. 6 and 7, could be used to align laser lights or other structures relative to the external apparatus so that a treatment could approach a planned target with its specific coordinates relative to the frame of the dental tray and, for example, the grids and the localizer as described above. Such an implementation might be particularly useful in treatment with a linear accelerator beam involving targets in the base of the skull, nose, throat, neck, and high thorax.

In this situation, the repeated identical orientation of the patient on the couch or table is important. Fractionated radiation, for example, could be implemented in several episodes on the same linear accelerator table with the patient oriented in the same position. The targets calculated for the irradiation could be calculated using the graphic reference means data with the localizer in place at the time of the image scanning. The specific details of the support bars can vary. It is envisioned that support bars can have adjustable joints and translatable slides so as to adjust the dental tray comfortably into the mouth of the patient and in an angular orientation of the head, which is desirable for the scanning and/or treatment.

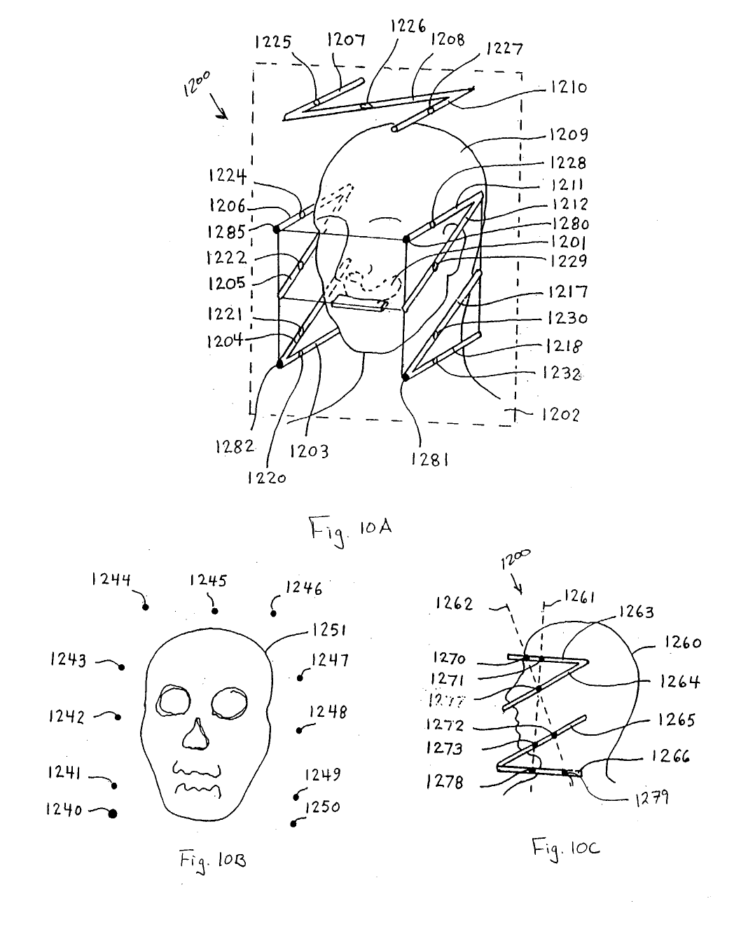

Turning now to FIGS. 9A-9C, a dental-based stereotactic localizer 100, in accordance with a further embodiment of the present disclosure, is shown. Stereotactic localizer 100 includes a dental tray 1101 having a platform 1120 attached thereto. Platform 1120 supports vertical elements 1110, 1111, 1116 and 1118, horizontal elements 1104, 1106, 1107 and 1109, and diagonal elements 1105, 1108, 1114 and 1115. Vertical elements 1110, 1111, 1116 and 1118, together with diagonal elements 1105, 1108, 1115 and 1114, give rise to varying index points in tomographic scans so as to determine a mapping from a two-dimensional slice image in an axial plane or quasi-axial plane to a three-dimensional coordinate system associated with dental tray 1101. Dental tray 1101 is directly molded to the teeth of the patient and thus is directly affixed to the skull of the patient via the upper and/or lower teeth of the patient. Horizontal rods 1104, 1106, 1107 and 1109, enable a quasi-coronal plane such as plane 1102 to include index data that in turn enables a mapping from a quasi-coronal slice through the face and related anatomy of the patient so as to map the data from this CT image slice to the coordinate reference frame associated with dental tray 1101.

FIG. 9B shows the index data as it might appear from a quasi-axial CT slice through the anatomy of the patient and through stereotactic localizer 100 of FIG. 9A. Index points from vertical elements 1110, 1111, 1116 and 1118 are shown by spots 1130, 1132, 1134 and 1133, respectively, while index marks from diagonal elements 1105, 1115, 1114 and 1108 are shown as spots 1131, 1133, 1139 and 1136, respectively. The anatomy of the patient is shown by outline 1137. The position of index points 1130, 1132, 1134, 1133, 1131, 1133, 1139 and 136 and their relative location enable such a two-dimensional planar data set as seen in FIG. 9B to be mapped into the three-dimensional coordinate system relative to dental tray 1101 and the attached platform 1120 according to the teachings of Brown.

Furthermore, FIG. 9C shows the index data as it might appear from a quasi-coronal CT slice 1102 through the anatomy of the patient and through stereotactic localizer 100. The index points from horizontal elements 1104, 1106, 1107 and 1109, are shown by spots 1155, 1153, 1150 and 1152, respectively, and index marks from diagonal elements 1105 and 1108 are shown as spots 1154 and 1151, respectively.

In particular, in FIG. 9C there is shown a slice through stereotactic localizer 100 which is not perfectly parallel to vertical elements 1110, 1111, 1116 and 1118. The fact that the lines associated with index point pairs 1150, 1152 and 1155, 1153 are not parallel indicates that the plane of the CT slice is not parallel to vertical elements 1110, 1111, 1116 and 1118. The location of index points 1150, 1152, 1155 and 1153 and the relationship of the angle included between the line element from 1150 to 1152, compared to the line element from 1153 to 1154, give further indication about the inclination of the plane of the CT slice relative to stereotactic localizer 100, and thus the mapping alluded to above can be done. Thus, each point of the anatomy, illustrated by 1157 of FIG. 9C, can be so mapped into the frame of reference of dental tray 101.

Turning now to FIGS. 10A-10C, another embodiment of a dental-based stereotactic localizer, in accordance with the present disclosure, is shown generally as 1200. Stereotactic localizer 1200 includes a dental tray 1201 attached cooperatively to rod and diagonal structures. In this case, the horizontal rod elements are illustrated by 1203, 1206, 1207, 1210, 1211 and 1218, and the diagonal rod elements are illustrated by 1204, 1205, 1208, 1212 and 1217. A mathematical plane, illustrated by the dashed plane 1202, is shown in a nearly coronal aspect to the face of the patient and intersects horizontal rod elements 1203, 1206, 1207, 1210, 1211 and 1218 at points 1220, 1224, 1225, 1227, 1228 and 1232, respectively, and intersects diagonal elements 1204, 1205, 1208, 1212 and 1217 at points 1221, 1222, 1226, 1229 and 1230, respectively.

FIG. 10B illustrates how these index points would look in a typical two-dimensional X-ray CT slice taken through plane 1202. Index marks for horizontal rod elements 1203, 1206, 1207, 1210, 1211 and 1218 are illustrated as spots 1240, 1243, 1244, 1246, 1247 and 1250, respectively, while the index points for diagonal rod elements 1204, 1205, 1208, 1212 and 1217 are illustrated as spots 1241, 1242, 1245, 1248 and 1249, respectively. The anatomy of the patient is illustrated by the diagram 1251.

By determining the two-dimensional coordinate positions of each of these index points, as well as any points in the anatomy of the patient in a two-dimensional CT slice, a transformation, calculation, or mapping can be made by computer, manually, or graphically between this data set and the three-dimensional coordinate reference frame of the dental tray and its associated attachment structure to the diagonal and horizontal rods. The pattern of the index points from the vertical rod elements and the diagonal rod elements, shown in FIG. 10B, make determinable not only the coordinate point mapping but also the inclination of plane 1202 of the CT slice.

FIG. 10C shows a side elevation view of the head of the patient with stereotactic localizer 1200 in place. Horizontal rod elements are illustrated by 1263 and 1266 while diagonal rod elements are illustrated by 1264 and 1265. While horizontal and diagonal rod elements are shown on one side of the head of the patient, it is understood that horizontal and diagonal rod elements can be provided on either side of the head of the patient. The projected planes of two CT slices are shown by 1262 and 1261. The intersection points for plane 1261 with horizontal rod elements 1263 and 1266 is at points 1271 and 1278, respectively, and with diagonal rod elements 1264 and 1265 is at points 1277 and 1273, respectively. Meanwhile, the intersection points for plane 1262 with horizontal rod elements 1263 and 1266 is at points 1270 and 1279, respectively, and with diagonal rod elements 1264 and 1265 is at points 1277 and 1272, respectively. The relationship of intersection points 1270, 1271, 1277, 1272, 1273, 1278 and 1279 can be seen to change, depending on the angle of plane 1262 and the position of plane 1261, and it is this kind of varying relationship of the intersection points, one relative to the other, which enables the full plane inclination angles as well as mapping of points within the plane to be done according to the Brown Patent. The use of horizontal rod element 1203, 1207, 1210, 1211 and 1218 of FIG. 10A and horizontal rod elements 1107, 1109, 1104 and 1106 of FIG. 9A, in conjunction with the diagonal rod elements makes it possible to do the mapping easily for an arbitrary CT slice plane.

Not shown in FIG. 10C are the intersection points for rod elements 1207, 1208 and 1210 with planes 1261 and 1262. These intersection points may also be used for an appropriate transformation of coordinates. With the use of two diagonal rod elements, namely, 1204, 1205 and 1212, 1217, as shown on each of the side structures of FIG. 10A, the top upper N-shaped structure gives added information which is duplicated but may improve accuracy. The upper N-shaped structure is not essential for these quasi-coronal slices, but is shown here for completeness, as it may be desired to have only one diagonal rod element on each of the structures to the left and right of the head of the patient. It should also be pointed out that not all of these horizontal rod elements and diagonal rod elements are necessary to do the mapping or transformation, nor is it necessary to do a full computer code matrix transformation to perform the mapping.

It is within the scope of the present disclosure that a simple triangular plate structure, a single horizontal and single diagonal rod element, varying numbers of horizontal and diagonal rod elements on the left, right, top, and other portions of the head, curvilinear rod or diagonal elements, spiral structures, arrays of index points, arrays of rod elements and pins of varying lengths and configurations could all be used to implement the basic disclosure of the dental-based stereotactic localizer disclosed herein. They are all embodiments of graphic reference means which may be attached to a dental reference frame that can be seen in tomographic or three-dimensional imaging processes that enable the frame of reference of the data from the image processing means to be transformed or mapped into the coordinate reference frame associated with the anatomy of the patient, in this case the skull, which is attached to the dental tray.

The devices such as those illustrated in FIGS. 9A-9C and 10A-10C are particularly useful in the ENT field, where quasi-coronal slices through the head of the patient are used. They provide an accurate and detailed mapping from the slice data to the anatomy of the patient which can then be used in turn to provide accurate approach, angulations, and calculation to targets and paths through the nose, mouth, or other facial aspects of the patient into interior anatomical target points for surgery. Use for maxofacial, nasal, pituitary, acoustic, head, throat, and larynx surgery is an obvious application. The extrapolation to stereotactic target coordination for radiation therapy is also a consequence and application. Furthermore, stereotactic localizer 1200 of FIG. 10A can include reference points or positions illustrated by 1282, 1285, 1280 and 1281, which are identifiable physical points of known or determinable coordinates associated with stereotactic localizer 1200 and that can be accessed during the imaging and also potentially during the surgical, set-up, or calibration phase of the treatment or surgery. As illustrated above, points 1282, 1285, 1280 and 1281 can be touched off by or located from, for example, a stereotactic digitizer or navigator which may be mechanical, optical, ultrasonic, radiofrequency, magnetic, or otherwise coupled to an interface to a computer graphic workstation, thereby enabling that navigator to be calibrated relative to the anatomy of the patient and recalibrated, if necessary, by simple insertion of the dental tray into the mouth of the patient.

Included within the scope of present disclosure is use of such identifiable physical points associated with the dental-based stereotactic localizers. Those skilled in the art can make further variations of these presently disclosed embodiments for the use in ENT surgery and surgery and therapy in and around the face. It should be noted that although the embodiments in FIGS. 9A-10C show what are called horizontal rod elements and diagonal rod elements thereto, it is possible to make such a localizer to function as described above by having only what one would say is diagonal rod elements; that is, rod elements that are angled one relative to the other, and not necessarily sets of parallel rod elements at all. For example, systems of V-shaped rod elements could be used or merely a single V-shaped structure (see FIG. 6B) could be used to give an index of the position of the tomographic slice. The combination of V-shaped or N-shaped structures, or other structures thereof, could be used in conjugation with additional knowledge about the orientation of the CT image plane such as its degree of parallelism to external apparatus to effect such a data mapping.

Having described the disclosure in certain embodiments, it is clear that those skilled in the art can make variations of the invention, and thus the embodiments above are meant only as illustrations.

What is claimed is:

1. A dental-based localizer for non-invasively indexing computer tomographic (CT) slice images of the anatomy of a patient, the localizer defining x, y and z-axes, whereby upon positioning of the localizer with respect to a head of the patient, the z-axis is in general parallel relation to the axis of the body, the localizer comprising:
a discrete independent reference structure which is solely supported by the patient, the reference structure including:
a dental piece configured and adapted to be received in the mouth of the patient;
a base plate attached to the dental piece, the base plate having a single monolithic element defining a general arcuate configuration, the arcuate configuration having a first portion including a first end and a second portion including a second end, wherein the first portion and the second portion of the base plate extend an approximately equal distance from the dental piece and terminate in the first end and the second end, respectively, the first end and the second end being positioned approximately adjacent the opposed posterior regions of the teeth of the patient, the dental piece providing a fixed reference point for the base plate relative to the patient, the dental piece and the base plate being configured and adapted for use on the patient without an external support structure attached thereto; and
a plurality of elongated graphic reference elements connected to the base plate and being disposed in at least two planes, the graphic reference elements and base plate being configured to accommodate the positioning of the patient in a supine position on a treatment table such that the occipital region of the head of the patient is directly positionable on the treatment table.

2. The dental-based localizer of claim 1, wherein the graphic reference elements includes an elongate diagonal reference element the at least two planes, each diagonal reference element extending in a direction having at least two axial components of direction with respect to the x, y and z axes.

3. The dental-based localizer of claim 2, further comprising a pair of parallel horizontal reference elements in each of the at least two planes, wherein each pair of horizontal reference elements is connected to a respective diagonal reference element.

4. The dental-based localizer of claim 1, wherein the elongated graphic reference elements are disposed in at least three planes, wherein adjacent planes intersect and are in oblique relation.

5. The dental-based localizer of claim 4, further comprising an elongated diagonal graphic reference element disposed in at least one of the at least three planes and extending in a direction having at least two axial components of directions with respect to the x, y and z axes.

6. The dental-based localizer of claim 5, further comprising a diagonal graphic reference element disposed in each of the at least three planes.

7. The dental-based localizer of claim 1, wherein the dental based localizer is fabricated from light weight materials such as medical grade plastics and graphics.

8. The dental-based localizer of claim 1, wherein the graphic reference element is disposed in at least one plane.

9. The dental-based localizer of claim 1, wherein the graphic reference element includes at least two diagonal graphic elements.

10. The dental-based localizer of claim 1, wherein the dental piece is adapted to be connected to a support bar.

11. The dental-based localizer of claim 1, wherein the graphic reference element provides contact points connected to the dental piece, which contact points are adapted to be physical reference points for calibrating a stereotactic device.

12. A dental based target localizing apparatus configured for use with an external treatment apparatus comprising:
a dental piece positionable in the mouth of a patient;
a base plate solely attached to the dental piece, the base plate defining an arcuate configuration and extending from the dental piece to posterior regions of the teeth of the patient, the dental piece and base plate providing a dental based reference, wherein an occipital region of the head of the patient is directly positionable on a treatment table;
a plurality of plates solely connected to the base plate; and
a reticule system positioned on the plurality of plates and adapted for aligning a target tissue portion in the patient with an external treatment apparatus for the treatment of the target tissue portion by the external treatment apparatus through the reticule system, wherein the base plate and the plurality of plates are configured to accommodate the positioning of the patient in a supine position on a treatment table such that the occipital region of the head of the patient is directly positionable on the upper surface of the treatment table.

13. The dental based target localizing apparatus of claim 12, wherein the reticule system includes a grid system.

14. The dental based target localizing apparatus of claim 12, wherein the reticule system includes graphics for quantifying distances in a standard unit of measure.

15. The dental-based target localizing apparatus of claim 14, wherein the reticule system includes indices configured as graphic reference means adapted for use with an external image scanning apparatus to identify a location of the target tissue portion in the patient using a three dimensional coordinate frame relative to the dental piece, the tissue target location being calculated by means of the imaging data from image slices through the anatomy of the patient and indices by the imaging scanning devices.

16. The dental based target localizing apparatus of claim 15, wherein the external treatment apparatus is a linear accelerator X-ray beam apparatus.

17. The dental based target localizing apparatus of claim 12, wherein the reticule system is adapted for use with a registration means of the external treatment apparatus.

18. The dental based target localizing apparatus of claim 12, wherein the reticule system is adapted for use with a laser system, the laser system being the external registration means for the external treatment apparatus.

19. The dental based target localizing apparatus of claim 15, wherein the indices are radiopaque.

20. The dental based target localizing apparatus of claim 12, wherein the reticule system includes indices and at least a portion of the indices are radiopaque.

21. The dental based target localizing apparatus of claim 12, wherein the dental piece includes a dental tray.

22. A method of identifying the location of specific anatomical points in a patient comprising the steps of:
providing a dental-based localizer including:
a dental piece configured and adapted to be received in the mouth of the patient;
a base plate attached to the dental piece, the base plate having a single monolithic element defining a general arcuate configuration, the arcuate configuration having a first portion including a first end and a second portion including a second end, wherein the first portion and the second portion of the base plate extend an approximately equal distance from the dental piece and terminate in the first end and the second end, respectively, the first end and the second end being positioned approximately adjacent the to opposed posterior regions of the teeth of the patient, the dental piece providing a fixed reference point for the base plate; and
a plurality of elongated graphic reference elements connected to the base plate and disposed in at least two planes, the graphic reference elements and the base support plate being adapted for use with a treatment table having a treatment surface, the graphic reference elements and base plate being configured to accommodate the positioning of the patient in a supine position such that the occipital region of the head of the patient is directly positionable on the surface of the treatment table without additional support for the head of the patient and without any connection of an external support structure to the graphic reference elements, base plate, and dental piece;
positioning the dental piece of the dental-based localizer in the mouth of the patient;
positioning the patient in the supine position on the treatment table such that the head of the patient is directly contacting the surface of the treatment table; and
performing an image scan of the head of the patient and the dental-based localizer, the dental based localizer providing a reference structure to index the data of the imaging scanner to the dental piece and the head of the patient.

23. The method of identifying the location of specific anatomical points of claim 22, wherein the step of positioning the dental piece also includes positioning a strap over a top of and around a portion of a back of the head of the patient.

24. The method of identifying the location of specific anatomical points of claim 22, wherein the dental-based localizer includes a plurality of plates positioned on the base plate, the plurality of plates having a reticule system and a plurality of graphic reference elements configured as indices.

25. The method of identifying the location of specific anatomical points of claim 24, wherein the step of imaging the head of the patient further includes identifying specific anatomical points in the patient, the plurality of plates being adapted for aligning the specific anatomical points and a treatment machine through the reticule system.

26. The method of identifying the location of specific anatomical points of claim 22, further comprising the step of providing graphic reference elements suitably positioned on the dental-based localizer for locating at least one of the cranium, skull base, nose, jaw and spine of the patient.

27. A dental-based localizer for locating targets in the anatomy of a patient in the region of the ear, nose, throat and skull base during tomographic coronal scanning in ENT surgery, the anatomy of the patient defining a longitudinal axis when in a supine position, the localizer comprising:
a dental piece configured to be positioned on the teeth of the patient to locate the localizer in fixed relation relative to the anatomy of the patient;
pairs of graphic reference elements positioned in at least two planes, each pair of graphic reference elements including at least one elongate element transversely oriented with respect to the longitudinal axis; and
at least one diagonal element having at least one end joined to an end of the at least one elongate element and oriented at an angle relative to the elongate element, the pairs of graphic reference elements and the dental piece being interconnected by an attachment structure having connecting members and supporting members.

28. The dental-based localizer of claim 27, wherein the at least two planes are parallel and are positioned along a respective side of the head of the patient when the dental piece is positioned on the teeth of the patient.

29. The dental-based localizer of claim 27, wherein graphic reference elements are positioned in a third plane, the graphic reference elements of the third plane are connected with at least one of the pairs of graphic reference elements positioned in the at least two planes.

30. A dental-based localizer for locating targets in the anatomy of a patient in the region of the ear, nose, throat and skull base during tomographic scanning, the anatomy of the patient defining a longitudinal axis while in a supine position, the localizer comprising:
a dental piece fit to the teeth of the patient thereby locating the dental piece in fixed spaced relation to the anatomy of the patient, the dental piece connected to an angled platform; and
graphic reference elements positioned relative to the platform, the graphic reference elements including:
a first graphic reference element positioned adjacent the head of the patient and defining a first plane, the first graphic reference element including at least one axial element oriented parallel to the longitudinal axis and at least one diagonal element oriented at an angle relative to the at least one axial element;
a second graphic reference element positioned adjacent the head of the patient and defining a second plane, the second graphic reference element including at least one axial element oriented parallel to the longitudinal axis and at least one diagonal element oriented at an angle relative to the at least one axial element of the graphic reference element, the at least one diagonal element of the first graphic reference element has components in axes x, y, and z which are perpendicular to components of the diagonal element of the second graphic reference element; and
a third graphic reference element defining a third plane, the third graphic reference element having a first diagonal element connected to the first graphic reference element and a second diagonal element connected to the second graphic reference element, the first and second diagonal elements of the third graphic reference element being joined at one end to one another.

31. The dental-based localizer of claim 30, wherein the first plane of the graphic reference element and the second plane of the second graphic reference element are converging planes.

32. The dental-based localizer of claim 30, wherein the first diagonal element of the third plane is connected to the first graphic reference element at an end opposite the joined end thereof and the second diagonal element of the third plane is connected to the second graphic reference element of an end opposite the joined end thereof.

33. The dental-based localizer of claim 30, wherein at least two of the planes have reference elements which are oriented horizontally and vertically with respect to the longitudinal axis.

34. A dental-based localizer that can be used for locating targets in the anatomy of a patient in the region of the ear, nose, throat and skull base, comprising:
a dental piece configured to be positioned on the teeth of the patient so as to locate thereby the dental piece to the anatomy of the patient, the dental piece being connected to a platform having a substantially plate shape and defining a dental plane; and
a graphic reference element attached to and located in position relative to the dental piece, the graphic reference element being connected to the platform and including:
two quadrangles supported on the platform, wherein a quadrangle is positioned adjacent a respective side of the head of the patient; and
a triangle interconnecting the two quadrangles and oriented in an inverted “V” shape, the two quadrangles and the triangle defining three interconnected planes extending from a top of the skull of the patient to a base of the throat of the patient, each plane having at least one diagonal element which will produce varying index data in a coronal tomographic slice from a computer tomographic imaging machine.

35. The apparatus of claim 34, wherein the at least one diagonal element comprises a plurality of diagonal elements which are oriented at an angle with respect to each other such that the index data includes patterns of fiducial points such that the distances between the fiducial points give position data that can be used to reference the position of the coronal tomographic slice relative to said dental piece, and therefore of the anatomy of the patient.

36. The apparatus of claim 34, wherein each of the quadrangles is defined by a pair of parallel rods, wherein the parallel rods are either in parallel alignment with a longitudinal axis defined by the anatomy of the patient when in a supine position or transverse to the longitudinal axis, and the at least one diagonal element includes at least one diagonal rod which is oriented at an angle relative to the parallel rods such that when the coronal tomographic slice intersects the parallel rods and the diagonal rod, the computer tomographic slice produces fiducial marks in the index data which can be used to reference the coronal tomographic slice with respect to the dental piece, and therefore with respect to the anatomy, which is included in the coronal tomographic slice, the anatomy including that near the face of the patient.

37. The apparatus of claim 36, wherein the at least one diagonal element comprises a plurality of diagonal elements, wherein the plurality of diagonal elements and the parallel rods provide fiducial marks in the index data for an arbitrary coronal tomographic slice through the graphic reference and the anatomy so that a mapping and/or transformation can be performed using the index data to relate a plurality of points on the anatomy of the patient, as seen in the arbitrary coronal tomographic slice, to the frame of reference of the dental piece and of the graphic reference.

38. The apparatus of claim 34, further comprising:
a stereotactic instrument with a pointing element cooperatively coupled to the stereotactic instrument so that the orientation and/or location of the pointing element relative to the stereotactic instrument is readable as stereotactic instrument data; and
physical locating points on the graphic reference element which are adapted to be coupled to and located at predetermined positions with respect to the dental piece and which are adapted to be located in space by the pointing element of the stereotactic instrument, whereby the stereotactic instrument data associated with the physical locating points is used to relate the position of the stereotactic instrument to the dental piece, and thereby to relate the stereotactic instrument to the anatomy of the patient as determined by the coronal tomographic slice.

39. A dental-based stereotactic localizer for non-invasively inducing computer tomographic (CT) slice images of the anatomy of a patient, wherein the anatomy of the patient defines a longitudinal axis when in a supine position, the localizer comprising:
a dental piece configured and adapted to be received in the mouth of the patient;
a support structure secured to the dental piece, the support structure defining an arcuate configuration which extends to the posterior margins of the teeth of the patient thus allowing an occipital region of the head of the patient to directly contact a surface of a treatment table; and
a plurality of graphic reference elements, wherein the plurality of graphic reference elements produce index information on an image scan when intersected by a plane of a scan slice, wherein the index information is used to develop a mapping of data from the image scan so as to reference the data from the image scan to a coordinate system of the dental tray.

40. The dental-based stereotactic localizer according to claim 39, wherein the plurality of graphic reference elements are parallel to one another.

41. The dental-based stereotactic localizer according to claim 40, wherein the plurality of graphic reference elements are oriented in a direction parallel to the longitudinal axis.

42. The dental-based stereotactic localizer according to claim 41, further including at least one index marker on each graphic reference element.

43. The dental-based stereotactic localizer according to claim 42, wherein the index markers are radiopaque markers located at specific locations along graphic reference elements.

44. The dental-based stereotactic localizer according to claim 40, further including at least one diagonal graphic reference element extending between adjacent parallel graphic reference elements.

45. The dental-based stereotactic localizer according to claim 44, wherein at least one of a distal and a proximal end of each diagonal graphic reference element is connected to adjacent parallel graphic reference elements.

46. The dental-based stereotactic localizer according to claim 45, wherein a distal end of each parallel graphic reference element includes a mechanical contact point.

47. The dental-based stereotactic localizer according to claim 46, wherein the mechanical contact point includes at least one of an indentation, a divot and a drill hole.

48. The dental-based stereotactic localizer according to claim 44, further including a stereotactic apparatus having a pointer element at a distal end thereof, wherein the pointer element is used to touch various points along at least one of the diagonal and parallel graphic reference elements to produce a mapping between a coordinate system of the stereotactic apparatus and a coordinate system of the stereotactic localizer.

49. The dental-based stereotactic localizer according to claim 43, further including at least one detecting apparatus configured and adapted to monitor the position of the index markers.

50. The dental-based stereotactic localizer according to claim 49, wherein the at least one detecting apparatus is configured and adapted to detect at least one LED disposed on the at least one graphic reference element.
